(19) United States
(12) Patent Application Publication
(10) Pub. No.: US 2009/0124019 A1
(43) Pub. Date: May 14, 2009

(54) ABSOLUTE METHOD FOR THE QUANTIFICATION OF ORGANIC COMPOUNDS

(75) Inventors: Jorge Ruiz Encinar, Oviedo (ES); Jose Ignacio Garcia Alonso, Oviedo (ES)

Correspondence Address: DARBY & DARBY P.C., P.O. BOX 770, Church Street Station, New York, NY, 10008-0770, US

(73) Assignee: UNIVERSIDAD DE OVIEDO, Oviedo (ES)

(21) Appl. No.: 12/089,803

(22) PCT Filed: Oct. 11, 2006
(86) PCT No.: PCT/ES2006/070153

§ 371 Date: Aug. 27, 2008

(30) Foreign Application Priority Data
Oct. 11, 2005 (ES) P200502590

(51) Int. Cl. G01N 33/00; G01N 30/02

(52) U.S. Cl. 436/146; 436/161

(57) ABSTRACT

The present invention relates to the quantification of organic compounds originating from a chromatography system that does not involve the use of patterns, comprising the continuous conversion of the carbon of the compounds into carbon dioxide (CO₂) involving a chemical reaction and the mixing of same, before or after separation, with a known, constant flow of a carbon compound, preferably CO₂, with carbon isotope-13 (¹³C). The mixture is introduced into a mass spectrometer in which the isotopic ratio ¹²C/¹³C is continuously measured either in the carbon or in a characteristic ion originating from same. The isotope ratio is converted into a carbon mass flow measurement using on-line isotope dilution.

This application is a U.S. national phase application under 35 U.S.C. §371 of International Patent Application No. PCT/ES2006/070153 filed Oct. 11, 2006, which claims the benefit of priority to Spanish Patent Application No. P200502590 filed Oct. 11, 2005, the disclosures of all of which are hereby incorporated by reference in their entireties. The International Application was published in Spanish on Apr. 19, 2007 as WO 2007/042597.

### FIELD OF THE INVENTION

The present invention relates to the field of instrumental analytical chemistry and, more specifically, relates to methods of analyzing organic compounds using chromatographic separation systems, without the need to calibrate the instrumental response of the specific equipment for each compound. Application of the present invention encompasses sectors such as the environmental sector and control of industrial processes.

### BACKGROUND

When it is desired to determine the content of one or several organic compounds in any sample arriving at an analysis laboratory, the following steps are generally followed:

a) If the sample is solid, the organic compounds to be analyzed are dissolved or lixiviated in a suitable solvent. In the case of liquid samples, all that is required is extraction of the organic compounds into a suitable solvent. At this time, or later, some type of internal standard could be added to the sample in order to correct subsequent methodological errors. These internal standards may be: i) a compound different from those that are to be analyzed and which is not present in the sample (internal standard method), ii) one or several of the compounds to be analyzed wherein the isotopic abundance of any of the elements present in the compound has been altered (isotope dilution method), or iii) one or several of the compounds to be analyzed, but without modifying the natural isotopic abundances (standard addition method). When the sample is liquid, the internal standard may be added directly onto the sample.

b) Once the compounds have been extracted from the solid, or when the sample is liquid, a process of pre-concentration or separation of other interfering compounds by means of non-chromatographic techniques may be necessary, the yield whereof must be determined.

c) Finally, the prepared sample is injected into a chromatography system in order to separate the different organic compounds present and detect them by means of a suitable instrumental device.

d) The signal, in peak area or peak height, is compared to a calibration line prepared from pure standards for each compound to be analyzed, since the instrument signal is different for each compound. This calibration makes it possible to determine the content of the organic compound in the injected sample. It is worth noting that this step of the analysis is, without a doubt, the costliest in the process, since certified standards are expensive and each compound present in the sample requires independent calibration, which requires time and money.

e) If some type of internal standard, amongst those described above, has been used, the quantification methodologies differ slightly.

Isotope dilution is a classical elemental determination technique described over fifty years ago. It is based on the deliberate alteration of the isotopic abundance of an element in the sample by the addition of a known quantity of the same element with an altered isotopic abundance (tracer) and measurement of the final isotopic abundance in the mixture by means of mass spectrometry.

This principle may be applied to the determination of the quantity of an element eluted from a chromatography system using on-line isotope dilution, the basic principle whereof has already been described in various publications [(1) to (5)]. The sample and the tracer are continuously mixed (in flow) following chromatographic separation and the isotope ratio in the mixture, Rm, is measured as a function of time by means of mass spectrometry. If the tracer input to the mixture is kept constant over time, the isotope ratio measured in the mixture (Rm) will depend solely on the element to be analysed in the sample, which may be constant or variable.

When on-line isotope dilution is used for elemental analysis following a chromatographic separation, it is essential for isotopic equilibrium to exist between the compounds eluted from the chromatography column and the chemical compound used as a tracer, which contains the isotopically-enriched element.

The achievement of this isotopic equilibrium has been described in the state of the art using inductively-coupled plasma as the ionisation source, since it is capable of breaking all the chemical bonds, in both the compounds from the chromatography column and the chemical compound containing the tracer [(1) to (5)]. Therefore, the effectiveness of the ionisation process will be independent from the nature of the chemical compound containing the analyte or the enriched tracer. For the determination of organic compounds by on-line isotope dilution, there is a publication in the state of the art that describes the use of inductively-coupled plasma as the ionisation source and ¹³C-labelled benzoic acid as the tracer (6). This ionisation source is not the most suitable for determining carbon, due to the low yield of the ionisation process for this element and potential contamination by atmospheric carbon.

### DETAILED DESCRIPTION OF THE INVENTION

The present invention relates to a method of quantifying organic compounds that eliminates and/or simplifies some of the steps known in the state of the art in an absolute determination method for organic compounds.

“Absolute determination” or “absolute method” means any determination or method performed without calibration, that is, without the need to use pure standards for each compound to be analysed.

One aspect of this invention provides a method of quantifying organic compounds in a sample, which comprises the following steps:
a. separating the compounds in the sample by means of chromatography;
b. the compounds separated in (a) are mixed with a tracer enriched in carbon-13;
c. the mixtures in section (b) chemically react, to produce CO₂;
d. measuring the isotope ratio of ¹²C/¹³C generated in (c) by means of a mass spectrometer, generating a ¹²C/¹³C isotope ratio chromatogram;
e. converting the ¹²C/¹³C isotope ratio chromatogram into a carbon mass flow chromatogram by means of the on-line isotope dilution equation;
   where, if the tracer is CO₂ enriched in carbon-13, mixing with the tracer may also be performed subsequent to the chemical reaction of the compounds separated in (a).

The method of this invention comprises a chemical reaction capable of quantitatively converting the carbon present in each organic compound eluted from the chromatography column into carbon dioxide (CO₂) as a means to achieve isotopic equilibrium. Where said chemical reactions may be:
a) a gas-phase combustion reaction using oxygen, oxygen-argon or oxygen-hydrogen mixtures;
b) a solid-phase oxidation reaction using metallic oxides (CuO, Ag₂O or others) in the presence of catalysts; or
c) a liquid-phase oxidation reaction using strong oxidants (permanganate, dichromate, persulfate or others).

The carbon dioxide obtained from the chemical reaction of each organic compound in the sample is continuously mixed with a carbon-13-enriched tracer prior to being introduced into the ionisation source of the mass spectrometer for on-line isotope dilution analysis.

The method of this invention to introduce the carbon isotopically labelled with ¹³C and perform the on-line isotope dilution comprises three equally valid alternative routes:

a) Introducing the tracer directly, as carbon dioxide enriched in ¹³C.

b) Introducing the tracer as another carbon compound (organic or inorganic, such as, for example, sodium carbonate) enriched in ¹³C dissolved in a liquid phase.

c) Introducing the tracer as another volatile carbon compound enriched in ¹³C, such as, for example, methane.

In the first case, that is, using CO₂ enriched in ¹³C as the tracer, the latter may be introduced into the chromatography system at any time (in the mobile phase, following the column or following the chemical reaction), whereas in the second and third cases, that is, when the tracer is a carbon compound other than CO₂, it must always be introduced prior to the chemical reaction in order to also convert the compound used as the tracer into CO₂ enriched in ¹³C.

In a preferred embodiment of the method of this invention, the mixture obtained, which is a mixture of the organic compound to be analysed and the tracer, is continuously introduced into a mass spectrometer in order to measure the carbon isotope ratio, ¹²C/¹³C, thus obtaining the mass flow chromatogram.

The input of the element into the mixture from the sample varies with time due to chromatographic separation and, therefore, the isotope dilution equations are modified by entering the mass flow of the element as the unknown variable [(1) to (5)]. The representation of the mass flow as a function of time will be the so-called mass flow chromatogram. The integration of each chromatographic peak provides an absolute measurement of the mass of the element in the peak. The concentration in the original sample may be calculated if the mass (or the volume) of sample injected into the chromatography system is known [(1) to (5)].

Measurement of the ¹²C/¹³C isotope ratio may be performed, depending on the ionisation source used, both in the monopositive ions of carbon with masses 12 and 13 and in other ions derived therefrom, such as, for example, in CO₂⁺ with masses 44 and 45, respectively.

The method of this invention makes it possible to achieve isotopic equilibrium between the compounds eluted from the chromatography column and the isotopically-enriched tracer prior to introduction into the ionisation source. The achievement of this prior isotopic equilibrium makes it possible to choose the most suitable ionisation source for the analysis of organic compounds, which could operate in high vacuum, thus providing greater sensitivity and lower carbon contaminations, which would translate into better detection limits for organic compounds.

Therefore, this invention provides a high-sensitivity, high-precision absolute determination method for organic compounds using on-line isotope dilution, through the continuous measurement of the ¹²C/¹³C isotope ratio at the outlet of a chromatography system using ionisation sources other than inductively-coupled plasma.

The absolute quantification method for organic compounds of this invention entails, with respect to the quantification methodologies for organic compounds described in the prior state of the art, the following advantages:

a) It is a universal method that could be used for any organic compound regardless of its chemical form, since the carbon present in any organic compound is susceptible to being quantitatively converted into CO₂ by means of one of the chemical reactions described above.

b) It is an absolute method that does not require using calibration standards for each compound to be determined, which significantly reduces the cost and the time required for analysis in routine laboratories. In fact, it would allow for the absolute quantification of any carbon-containing compound in the sample eluted from the chromatography system, with a single injection and without the need for any standard whatsoever.

c) It uses isotope dilution and, therefore, it may be considered to be a primary analysis method that may be directly traceable to the International System of Units.

d) It requires minimal modification of current chromatography systems in order to introduce the chemical reaction and the flow of enriched carbon 13.

e) It uses low-cost mass spectrometers, since high precisions are not required when measuring the isotope ratios.

f) It makes it possible to use internal standards to correct methodological errors during preparation of the sample, variations in the volume injected or variations in volume due to evaporation of the solvent, since this methodology makes it possible to also absolutely determine the quantity of internal standard injected.

Another aspect of this invention is the application of the method described, in both liquid-chromatography separations and gas-chromatography separations, in environmental assay laboratories and industrial process control laboratories where routine analyses of organic compounds with known chemical structures are performed by means of chromatographic techniques.

Specifically, the determination of aliphatic hydrocarbons in various types of samples, of aromatic hydrocarbons, pesticides, organochlorated compounds, etc. would be feasible with this invention. Therefore, one of the most immediate fields of application is environmental analysis of organic pollutants in assay laboratories. A second important field of application would be in the quality control of industrial and pharmaceutical products and the control of chemical processes, which are normally conducted in laboratories in the production plants themselves.

Other fields of application, within analytical chemistry, could be the optimisation and study of sample preparation processes (liquid-liquid extraction, solid-liquid extraction, solid-phase microextraction, etc.), since the absolute quantification performed with this method may be used to study and optimise this process in a highly reliable manner. A specific case is the selection and use of internal standards to correct errors in separation processes.

From an instrumental standpoint, this invention can be incorporated into chromatography equipment that uses mass spectrometry as the detection system (simple GC-MS or HPLCMS equipment) and, therefore, consists of an instrumental modification susceptible to being commercialised by analytical instrumentation companies.

This modified equipment would make it possible to perform routine analyses of organic compounds with high precision and accuracy, at a low cost for laboratories and in a considerably shorter period of time than with traditional external calibration and standard addition methods.

Throughout the description and the claims, the word “comprises” and its variants are not intended to exclude other technical characteristics, components or steps. For those skilled in the art, other objects, advantages and characteristics of the invention will emerge partly from the description and partly from the practise of the invention. The following examples and drawings are provided for illustrative purposes and are not intended to limit this invention.

### BRIEF DESCRIPTION OF THE DRAWINGS

FIG. 1.—Illustration of the basic principle of isotope dilution analysis, where: “I.a.” is isotopic abundance; “S” is sample; “Mix.” is mixture; “T” is tracer; “a” is isotope a; and “b” is isotope b.

FIG. 2.—Illustration of the principle of on-line isotope dilution for a liquid chromatography system, where: “HPLC P.” is HPLC pump; “M. ph.” are mobile phases; “S.” is syringe; “I.v.” is injection valve; “C.” is column; “E.t.” is enriched tracer; “P.p.” is peristaltic pump; and “I.s. (M.S.)” is the ionisation source of the Mass Spectrometer.

FIG. 3.—Diagram of a preferred embodiment in Gas Chromatography, where: “C.int.” is combustion interface; “C.inj.” is chromatography injector; “C.G.” is Gas Chromatography; “J” is joint; “M.S.” is Mass Spectrometer; “(E.i.) I.s.” is electronic-impact ionisation source; “C.c.” is chromatography column; “M.f.c.” is mass flow controller; and “Res. ¹³CO2 (He)” is reservoir of CO₂ enriched in ¹³C.

### EXAMPLE

For a better understanding of the method described in this invention, we will explain the procedure to be followed in order to analyze organic compounds by gas chromatography using a quadrupole mass spectrometer with an electronic-impact ionisation source.

The basic principle of isotope dilution analysis is illustrated in FIG. 1 for an element having two isotopes: a and b. As can be observed, isotope a is the most abundant in the sample, whilst the tracer is enriched in isotope b. The isotopic abundance in the sample will be a linear combination of the abundances in the sample and the tracer, which will depend on the quantity of the element present in the sample and the quantity of tracer added. Measurement of the isotope ratio, Rm, in the sample (isotope a/isotope b) makes it possible to determine the quantity of the element in the original sample.

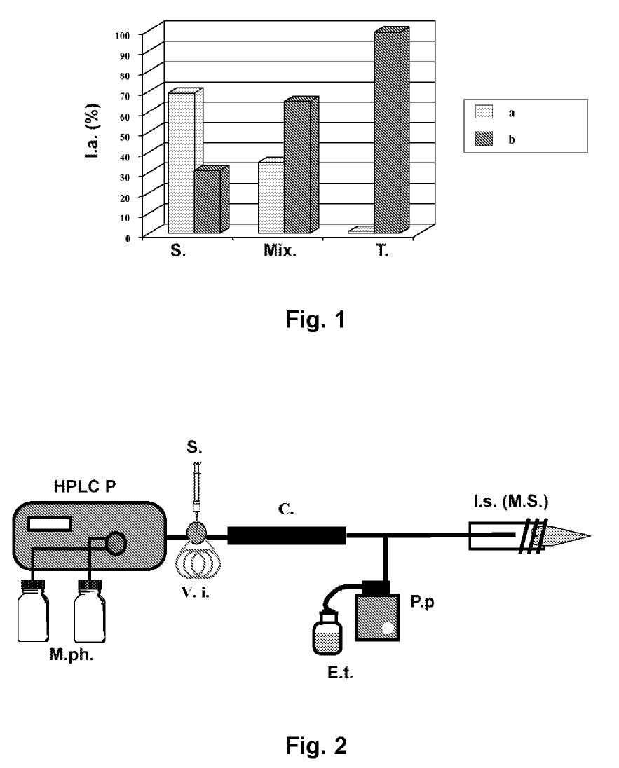

This basic principle may be applied to the determination of the quantity of an element eluted from a chromatography system using on-line isotope dilution, the basic principle whereof is illustrated in FIG. 2 for a liquid chromatography system. As can be observed in FIG. 2, the sample and the tracer are continuously mixed (in flow) following chromatographic separation and the isotope ratio in the mixture, Rm, is measured as a function of time by means of mass spectrometry.

The carbon dioxide enriched in ¹³C was prepared dissolved in pressurised helium. The container with the tracer had an output opening and the gas flow was controlled by means of a mass flow controller that exactly regulated the flow of tracer added to the mixture. On the other hand, a combustion interface was installed at the outlet of the chromatography column, consisting of a ceramic tube wherein copper threads, previously oxidised to CuO in an air current, were introduced and maintained at a suitable temperature (>850° C.) in order for the following generic reaction to take place quantitatively:

CₙH₂ₙ₊₂+(3n+1)CuO->nCO₂+(n+1)H₂O+(3n+1)Cu

where CₙH₂ₙ₊₂ was any aliphatic hydrocarbon.

Any organic compound would produce the previous reaction quantitatively, being converted into carbon dioxide, water and, in the presence of heteroatoms, other reaction products (NO₂, SO₂, etc).

The flow of ¹³C-labelled carbon dioxide was added at the outlet of the combustion interface and the mixture thus generated was introduced, through a fused silica tube, into the ionisation source of a mass spectrometer.

Using an electronic-impact ionisation source, it was possible to measure the carbon isotope ratios in the CO₂⁺ molecular ion with masses 44 (for carbon 12) and 45 (for carbon 13). The measured isotope ratio was converted into a ¹²C/¹³C isotope ratio by taking into account oxygen's natural isotopic abundances.

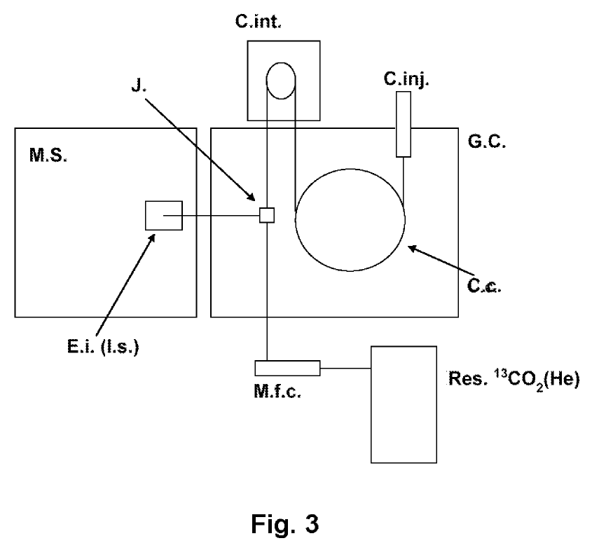

This isotope ratio was inserted into the on-line isotope dilution equation (1) to obtain the mass flow chromatogram and directly determine the carbon mass in each chromatographic peak following the corresponding integration. A general diagram of this embodiment is shown in FIG. 3.

### BIBLIOGRAPHICAL REFERENCES

(1) “Isotope dilution analysis for trace element speciation. A tutorial review”. Rodríguez González, P; Marchante Gayón, J M; García Alonso, J I; Sanz Medel, A. SPECTROCHIMICA ACTA PART B-ATOMIC 10 SPECTROSCOPY, 2005, 60 (2): 151-207.
(2) “Isotope-dilution mass spectrometry”. Heumann, K G. INTERNATIONAL JOURNAL OF MASS SPECTROMETRY AND ION PROCESSES, 1992, 118:575-592.
(3) “Speciation of essential elements in human serum using anion-exchange chromatography coupled to post-column isotope dilution analysis with double focusing ICP-MS”. Muñiz, CS, Marchante Gayón, J M; García Alonso, J I; Sanz Medel, A. JOURNAL OF ANALYTICAL ATOMIC SPECTROSCOPY, 2001, 16 (6): 587-592.
(4) “Quantitative speciation of selenium in human serum by affinity chromatography coupled to post-column isotope dilution analysis ICP-MS”. Reyes, LH, Marchante-Gayón, J M, García Alonso, J I; Sanz Medel, A. JOURNAL OF ANALYTICAL ATOMIC SPECTROSCOPY, 2003, 18 (10):1210-1216.
(5) “Determination of cadmium in biological and environmental materials by isotope dilution inductively coupled plasma mass spectrometry: Effect of flow sample introduction methods”. Mota, JPV, De la Campa, MRF, García Alonso, J I; Sanz Medel, A. JOURNAL OF ANALYTICAL ATOMIC SPECTROSCOPY, 1999, 14 (2): 113-120.
(6) “Development of an ICP-IDMS method for dissolved organic carbon determinations and its application to chromatographic fractions of heavy metal complexes with humic substances”. Vogl, J; Heumann, KG. ANALYTICAL CHEMISTRY, 1998, 70 (10): 2038-2043.

1. A method for absolute quantification of organic compounds in a sample, comprising the steps of:
a. separating the compounds in the sample by means of chromatography;
b. mixing the compounds separated in step (a) in continuous flow with a non-specific tracer enriched in carbon-13;
c. chemically reacting the compounds and the tracer mixed in step (b) to produce CO₂;
d. measuring the isotope ratio of ¹²C/¹³C generated in step (c) by means of a mass spectrometer, and generating a ¹²C/¹³C isotope ratio chromatogram; and
e. converting the ¹²C/¹³C isotope ratio chromatogram into a carbon mass flow chromatogram by means of an on-line isotope dilution equation.

2. The method of claim 1, wherein the tracer is a carbon inorganic compound.

3. The method of claim 2, wherein the tracer is sodium carbonate.

4. A method for absolute quantification of organic compounds in a sample, comprising the steps of:
a. separating the compounds in the sample by means of chromatography;
b. mixing the compounds separated in step (a);
c. chemically reacting the compounds in step (b) to produce CO₂;
d. mixing the reacted compounds in continuous flow with a CO₂ tracer enriched in carbon-13;
e. measuring the isotope ratio of ¹²C/¹³C generated in step (d) by means of a mass spectrometer, and generating a ¹²C/¹³C isotope ratio chromatogram; and
f. converting the ¹²C/¹³C isotope ratio chromatogram into a carbon mass flow chromatogram by means of an on-line isotope dilution equation.

5. The method of claim 1, wherein the tracer is methane.

6. The method of claim 1, wherein the separation in step (a) is performed by means of liquid chromatography or gas chromatography.

7. The method of claim 1, wherein the ¹²C/¹³C isotope ratio is measured directly.

8. The method of claim 1, wherein the isotope ratio is measured by means of ions derived from ¹²C and ¹³C.

9. The method of claim 8, wherein the ions derived from the ¹²C/¹³C ratio are CO₂⁺ molecular ions with masses 44 and 45, respectively.

10. The method of claim 1, wherein the ionisation source of the mass spectrometer is an electronic-impact ionisation source.

11. The method of claim 1, wherein the tracer is CO₂.
